SEQUENCE LISTING (28 sequences)

SEQ ID NO: 1 - protein, 56 aa, Streptococcus sp. GX7809
TTYKLILNGKTLKGETTTEAVDAATAEKVFKQYANDNGVDGEWTYDDATKTFTVTE

SEQ ID NO: 2 - protein, 56 aa, Streptococcus sp. GX7809
TTYKLVINGKTLKGETTTKAVDAETAEKAFKQYANDNGVDGVWTYDDATKTFTVTE

SEQ ID NO: 3 - protein, 56 aa, Unknown
  SpG Mutant
TTYKLVINGKTLKGETTTKAVDAETAERAFRQYATDNGVEGMWTYDPATKTFTVTE

SEQ ID NO: 4 - protein, 56 aa, Streptococcus sp. G148
TTYKLVINGKTLKGETTTEAVDAATAEKVFKQYANDNGVDGEWTYDDATKTFTVTE

SEQ ID NO: 5 - DNA, 171 bp, Artificial Sequence
  Synthetic  DNA code
accacctacaaactggtcatcaacggtaagaccctgaaaggtgaaaccaccaccaaagctgttgacgctgagacggccgaacgagcgttccgacagtacgctaccgacaacggtgtcgaaggtatgtggacctacgaccccgctaccaaaaccttcacggttaccgaatag

SEQ ID NO: 6 - DNA, 60 bp, Artificial Sequence
  Synthetic  oligo DNA
ctgggatccaccacctacaaactggtcatcaacggtaagaccctgaaaggtgaaaccacc

SEQ ID NO: 7 - DNA, 60 bp, Artificial Sequence
  Synthetic  oligo DNA
ttcggccgtctcagcgtcaacagctttggtggtggtttcacctttcagggtcttaccgtt

SEQ ID NO: 8 - DNA, 70 bp, Artificial Sequence
  Synthetic  oligo DNA
agacggccgaacgagcgttccgacagtacgctaccgacaacggtgtcgaaggtatgtggacctacgaccc

SEQ ID NO: 9 - DNA, 70 bp, Artificial Sequence
  Synthetic  oligo DNA
ccgggaattctattcggtaaccgtgaaggttttggtagcggggtcgtaggtccacataccttcgacaccg

SEQ ID NO: 10 - DNA, 171 bp, Artificial Sequence
  Synthetic  DNA code
accacctacaaactgatcctgaacggtaagaccctgaaaggtgaaaccaccaccgaagctgttgacgctgctacggccgaaaaagtgttcaaacagtacgctaacgacaacggtgtcgacggtgaatggacctacgacgacgctaccaaaaccttcacggttaccgaatag

SEQ ID NO: 11 - DNA, 171 bp, Artificial Sequence
  Synthetic  DNA code
accacctacaaactggtcatcaacggtaagaccctgaaaggtgaaaccaccaccaaagctgttgacgctgagacggccgaaaaagcgttcaaacagtacgctaacgacaacggtgtcgacggtgtatggacctacgacgacgctaccaaaaccttcacggttaccgaatag

SEQ ID NO: 12 - DNA, 171 bp, Artificial Sequence
  Synthetic  DNA code
accacctacaaactggtcatcaacggtaagaccctgaaaggtgaaaccaccaccgaagctgttgacgctgctacggccgaaaaagtgttcaaacagtacgctaacgacaacggtgtcgacggtgaatggacctacgacgacgctaccaaaaccttcacggttaccgaatag

SEQ ID NO: 13 - DNA, 22 bp, Artificial Sequence
  Synthetic  oligo DNA
ggatccaccagatacaaactgg

SEQ ID NO: 14 - DNA, 22 bp, Artificial Sequence
  Synthetic  oligo DNA
ccagtttgtatctggtggatcc

SEQ ID NO: 15 - DNA, 29 bp, Artificial Sequence
  Synthetic  oligo DNA
gaccctgaaaggttataccaccaccaaag

SEQ ID NO: 16 - DNA, 29 bp, Artificial Sequence
  Synthetic  oligo DNA
ctttggtggtggtataacctttcagggtc

SEQ ID NO: 17 - DNA, 29 bp, Artificial Sequence
  Synthetic  oligo DNA
gaccctgaaaggttggaccaccaccaaag

SEQ ID NO: 18 - DNA, 29 bp, Artificial Sequence
  Synthetic  oligo DNA
ctttggtggtggtccaacctttcagggtc

SEQ ID NO: 19 - protein, 56 aa, Unknown
  SpG Mutant
TRYKLVINGKTLKGETTTKAVDAETAERAFRQYATDNGVEGMWTYDPATKTFTVTE

SEQ ID NO: 20 - DNA, 171 bp, Artificial Sequence
  Synthetic  DNA code
accagatacaaactggtcatcaacggtaagaccctgaaaggtgaaaccaccaccaaagctgttgacgctgagacggccgaacgagcgttccgacagtacgctaccgacaacggtgtcgaaggtatgtggacctacgaccccgctaccaaaaccttcacggttaccgaatag

SEQ ID NO: 21 - protein, 56 aa, Unknown
  SpG Mutant
TTYKLVINGKTLKGYTTTKAVDAETAERAFRQYATDNGVEGMWTYDPATKTFTVTE

SEQ ID NO: 22 - DNA, 171 bp, Artificial Sequence
  Synthetic  DNA code
accacctacaaactggtcatcaacggtaagaccctgaaaggttataccaccaccaaagctgttgacgctgagacggccgaacgagcgttccgacagtacgctaccgacaacggtgtcgaaggtatgtggacctacgaccccgctaccaaaaccttcacggttaccgaatag

SEQ ID NO: 23 - protein, 56 aa, Unknown
  SpG Mutant
TTYKLVINGKTLKGWTTTKAVDAETAERAFRQYATDNGVEGMWTYDPATKTFTVTE

SEQ ID NO: 24 - DNA, 171 bp, Artificial Sequence
  Synthetic  DNA code
accacctacaaactggtcatcaacggtaagaccctgaaaggttggaccaccaccaaagctgttgacgctgagacggccgaacgagcgttccgacagtacgctaccgacaacggtgtcgaaggtatgtggacctacgaccccgctaccaaaaccttcacggttaccgaatag

SEQ ID NO: 25 - protein, 56 aa, Unknown
  SpG Mutant
TTYKLVINGKTLKGWTTTKAVDAETAEKAFKQYANDNGVDGVWTYDDATKTFTVTE

SEQ ID NO: 26 - DNA, 171 bp, Artificial Sequence
  Synthetic  DNA code
accacctacaaactggtcatcaacggtaagaccctgaaaggttggaccaccaccaaagctgttgacgctgagacggccgaaaaagcgttcaaacagtacgctaacgacaacggtgtcgacggtgtatggacctacgacgacgctaccaaaaccttcacggttaccgaatag

SEQ ID NO: 27 - protein, 56 aa, Unknown
  SpG Mutant
TRYKLVINGKTLKGYTTTKAVDAETAERAFRQYATDNGVEGMWTYDPATKTFTVTE

SEQ ID NO: 28 - DNA, 171 bp, Artificial Sequence
  Synthetic  DNA code
accagatacaaactggtcatcaacggtaagaccctgaaaggttataccaccaccaaagctgttgacgctgagacggccgaacgagcgttccgacagtacgctaccgacaacggtgtcgaaggtatgtggacctacgaccccgctaccaaaaccttcacggttaccgaatag

(19) United States
(12) Patent Application Publication
(10) Pub. No.: US 2017/0305965 A1
(43) Pub. Date: Oct. 26, 2017

(54) Fab REGION-BINDING PEPTIDE

(71) Applicant: KANEKA CORPORATION, Osaka-shi, Osaka (JP)

(72) Inventor: Shinichi YOSHIDA, Takasago-shi (JP)

(73) Assignee: KANEKA CORPORATION, Osaka-shi, Osaka (JP)

(21) Appl. No.: 15/503,511

(22) PCT Filed: Aug. 27, 2015
(86) PCT No.: PCT/JP2015/074265

§ 371 Date: Feb. 13, 2017

(30) Foreign Application Priority Data
Aug. 28, 2014 (JP) 2014-174074

(51) Int. Cl. C07K 1/22; C07K 16/32; C07K 14/315; B01D 15/34; C07K 17/00; B01D 15/38

(52) U.S. Cl. CPC C07K 1/22; C07K 17/00; B01D 15/3809; C07K 14/315; B01D 15/34; C07K 16/32; C07K 2317/24; C07K 2317/55; C07K 2317/92

(57) ABSTRACT

The objective of the present invention is to provide a Fab region-binding peptide which is excellent in a binding capability to a Fab region of IgG, an affinity separation matrix which has the peptide as a ligand, and a method for producing a Fab region-containing protein by using the affinity separation matrix. In addition, the objective of the present invention is to provide a DNA which encodes the peptide, a vector which contains the DNA, and a transformant which is transformed by the vector. The above-described problems can be solved by utilizing a Protein G variant having the mutation of an amino acid substitution at the specific position.

### TECHNICAL FIELD

The present invention relates to a Fab region-binding peptide which is excellent in a binding capability to a Fab region of an immunoglobulin G, an affinity separation matrix which has the peptide as a ligand, a method for producing a Fab region-containing protein by using the affinity separation matrix, a DNA which encodes the peptide, a vector which contains the DNA, and a transformant which is transformed by the vector.

### BACKGROUND ART

As one of important functions of a protein, a capability to specifically bind to a specific molecule is exemplified. The capability plays an important role in an immunoreaction and signal transduction in a living body. A technology utilizing the capability for purifying a useful substance has been actively developed. As one example of a protein which is actually utilized industrially, for example, Protein A affinity separation matrix has been used for capturing an antibody drug to be purified at one time from a culture of an animal cell with high purity (Non-patent Documents 1 and 2). Hereinafter, Protein A is abbreviated as “SpA” in some cases.

Most of antibody drugs launched presently are an immunoglobulin G. Hereinafter, an immunoglobulin G is abbreviated as “IgG” in some cases. In addition, an antibody drug consisting of an antibody fragment has been actively in clinical development. An antibody fragment is an antibody derivative having a molecular structure obtained by fragmenting IgG. A plurality of antibody drugs consisting of a Fab fragment of IgG has been launched (Non-patent Document 3).

In an initial purification step in an antibody drug production, the above-described SpA affinity separation matrix is utilized. However, SpA is basically a protein which specifically binds to a Fc region of IgG. Thus, SpA affinity separation matrix cannot capture an antibody fragment which does not contain a Fc region. Accordingly, an affinity separation matrix capable of capturing an antibody fragment which does not contain a Fc region of IgG is highly required industrially.

A plurality of proteins which can bind to a region except for a Fc region of IgG have been already known (Non-patent Document 4). However, it is not true that an affinity separation matrix having such a protein as a ligand is generally industrially utilized in purification of an antibody drug similarly to SpA affinity separation matrix.

For example, a protein referred to as Protein G found in Streptococcus sp. classified into Group G can bind to IgG. Hereinafter, Protein G is referred to as “SpG”. A SpG affinity separation matrix product on which SpG is immobilized as a ligand has been commercially available (product name: “Protein-G Sepharose 4 Fast Flow” manufactured by GE Healthcare, Patent Document 1). It is known that SpG strongly binds to a Fc region of IgG and weakly binds to a Fab region (Non-patent Documents 4 and 5). However, since SpG has a weak binding force to a Fab region, the performance of a SpA affinity separation matrix product to maintain an antibody fragment which does not contain a Fc region and which contains a Fab region only is considered to be insufficient. It was therefore promoted to improve the binding force of SpG to a Fab region by introducing a mutation into SpG (Patent Document 2).

### PRIOR ART DOCUMENT

### Patent Document

Patent Document 1: JP S63-503032 A
Patent Document 2: JP 2009-195184 A

### Non-Patent Document

Non-patent Document 1: Hober S. et al., J. Chromatogr. B, 2007, vol. 848, pp. 40-47
Non-patent Document 2: Shukla A. A. et al., Trends Biotechnol., 2010, vol. 28, pp. 253-261
Non-patent Document 3: Nelson A. N. et al., Nat. Biotechnol., 2009, vol. 27, pp. 331-337
Non-patent Document 4: Bouvet P. J., Int. J. Immunopharmac., 1994, vol. 16, pp. 419-424
Non-patent Document 5: Derrick J. P., Nature, 1992, vol. 359, pp. 752-754

### DISCLOSURE OF THE INVENTION

### Problems to be Solved by the Invention

As described above, Protein A (SpA) has been conventionally put to practical use as an affinity ligand to adsorb an antibody to be purified, but SpA has a specific affinity for only a Fc region of IgG. However, a ligand which also has an affinity for a Fab region is recently required, since a technology to use an antibody fragment as a drug has been developed. Protein G (SpG) is known as a protein having an affinity for not only a Fc region but also a Fab region, but an affinity for a Fab region thereof is lower than that for a Fc region. It is therefore examined to improve an affinity for a Fab region by introducing a mutation into SpG as the invention described in Patent Document 2. However, the SpG variant described in Patent Document 2 is not considered to have a sufficient affinity as a ligand for purifying an antibody fragment which does not contain a Fc region and which is composed of a Fab region, since the affinity of the SpG variant for a Fab region is merely higher about 2 times than that before introducing the mutation.

Under the above-described circumstances, the objective of the present invention is to provide a Fab region-binding peptide which is excellent in a binding capability to a Fab region of IgG, an affinity separation matrix which has the peptide as a ligand, and a method for producing a Fab region-containing protein by using the affinity separation matrix. In addition, the objective of the present invention is to provide a DNA which encodes the peptide, a vector which contains the DNA, and a transformant which is transformed by the vector.

### Means for Solving the Problems

The inventors of the present invention made extensive studies to solve the above problems. As a result, the inventors completed the present invention by finding that a structural energy of a SpG-IgG complex is amazingly stabilized, in other words, the binding between the two enjoys an advantage, by substituting the amino acid at the 2^{nd} position, which is far from the interaction interface with a Fab region, and the amino acid at the 15^{th} position, which is relatively close to but not the closest to the interaction interface with a Fab region, with the specific amino acids.

Hereinafter, the present invention completed as a result is described.

[1] A Fab region-binding peptide, selected from the following (1) to (3):

(1) a Fab region-binding peptide comprising an amino acid sequence of SEQ ID NO: 1 derived from β1 domain of protein G, an amino acid sequence of SEQ ID NO: 2 derived from β2 domain of protein G or an amino acid sequence of SEQ ID NO: 3 derived from β2 domain variant of protein G, having one or more amino acid substitution mutations selected from a mutation of substituting the amino acid residue at the 2^{nd} position with Arg and a mutation of substituting the amino acid residue at the 15^{th} position with Tyr or Trp;

(2) a Fab region-binding peptide comprising an amino acid sequence specified in the (1) having deletion, substitution and/or addition of one or more amino acid residues in a region except for the 2^{nd} position and the 15^{th} position, and having higher binding force to a Fab region of an immunoglobulin G than a binding force before introducing the amino acid substitution mutation;

(3) a Fab region-binding peptide comprising an amino acid sequence having a sequence identity of 80% or more with the amino acid sequence specified in the (1), and having higher binding force to a Fab region of an immunoglobulin G than a binding force before introducing the amino acid substitution mutation, provided that the amino acid substitution mutation specified in the (1) at one or more positions selected from the 2^{nd} position and the 15^{th} position is not further mutated in (3).

[2] The Fab region-binding peptide according to the above [1], wherein a position of the substitution is one or more positions selected from the 6^{th} position, the 7^{th} position, the 10^{th} position, the 13^{th} position, the 18^{th} position, the 19^{th} position, the 21^{st} position, the 24^{th} position, the 28^{th} position, the 29^{th} position, the 30^{th} position, the 31^{st} position, the 33^{rd} position, the 35^{th} position, the 39^{th} position, the 40^{th} position, the 42^{nd} position and the 47^{th} position in the amino acid sequence specified in the (2).

[3] The Fab region-binding peptide according to the above [1] or [2], wherein a position of the deletion and/or addition is N-terminal and/or C-terminal in the amino acid sequence specified in the (2).

[4] The Fab region-binding peptide according to any one of the above [1] to [3], wherein the sequence identity is 90% or more in the amino acid sequence specified in the (3).

[5] The Fab region-binding peptide according to any one of the above [1] to [4], wherein the amino acid residue at the 15^{th} position is substituted with Tyr in the amino acid sequence specified in the (1).

[6] A Fab region-binding peptide multimer, comprising two or more Fab region-binding peptides according to any one of the above [1] to [5] as domains, wherein the Fab region-binding peptides are connected one another.

[7] An affinity separation matrix, wherein the Fab region-binding peptide according to any one of the above [1] to [5] or the Fab region-binding peptide multimer according to the above [6] is immobilized on a water-insoluble carrier.

[8] A method for producing a protein comprising a Fab region, comprising the steps of:

contacting a liquid sample comprising the protein comprising the Fab region with the affinity separation matrix according to the above [7]; and

separating the protein comprising the Fab region adsorbed on the affinity separation matrix from the affinity separation matrix.

[9] A DNA, encoding the Fab region-binding peptide according to any one of the above [1] to [5].

[10] A vector, comprising the DNA according to the above [9].

[11] A transformant, transformed by the vector according to the above [10].

### Effect of the Invention

The Fab region-binding peptide according to the present invention has sufficiently high affinity for a Fab region of IgG, and therefore has affinity for not only a general antibody but also an antibody fragment which contain a Fab region but which does not contain a Fc region. As a result, an antibody fragment drug can be efficiently purified by using the affinity separation matrix obtained by immobilizing the Fab region-binding peptide according to the present invention on a water-insoluble carrier. Thus, the present invention is industrially very useful, since the present invention can contribute to the practical realization of an antibody fragment drug under the situation that an antibody fragment drug has been actively developed recently due to the low production cost or the like.

### BRIEF DESCRIPTION OF THE DRAWINGS

FIG. 1 represents a method for preparing an expression plasmid of a wild Protein G domain.

FIG. 2 are a bonding response curve charts of the Protein G domain before introducing the mutation and the Protein G domain variant according to the present invention to a Fab region of anti-HER2 monoclonal antibody. In FIG. 2, data of 0.1 μM, 0.2 μM, 0.4 μM and 0.8 μM of each peptide are demonstrated together.

FIG. 3 is a bonding response curve chart of the Protein G domain before introducing the mutation and the Protein G domain variants according to the present invention to a Fab region of anti-HER2 monoclonal antibody.

### MODE FOR CARRYING OUT THE INVENTION

The Fab region-binding peptide according to the present invention is any one of the following (1) to (3).

Fab Region-Binding Peptide (1)

A Fab region-binding peptide comprising an amino acid sequence of SEQ ID NO: 1 derived from β1 domain of protein G, an amino acid sequence SEQ ID NO: 2 derived from β2 domain of protein G or an amino acid sequence SEQ ID NO: 3 derived from β2 domain variant of protein G, having one or more amino acid substitution mutations selected from a mutation of substituting the amino acid residue at the 2^{nd} position with Arg and a mutation of substituting the amino acid residue at the 15^{th} position with Tyr or Trp;

Fab Region-Binding Peptide (2)

A Fab region-binding peptide comprising an amino acid sequence specified in the (1) having deletion, substitution and/or addition of one or more amino acid residues in a region except for the 2^{nd} position and the 15^{th} position, and having higher binding force to a Fab region of an immunoglobulin G than a binding force before introducing the amino acid substitution mutation;

Fab Region-Binding Peptide (3)

A Fab region-binding peptide comprising an amino acid sequence having a sequence identity of 80% or more to the amino acid sequence specified in the (1), and having higher binding force to a Fab region of an immunoglobulin G than a binding force before introducing the amino acid substitution mutation, provided that the amino acid substitution mutation specified in the (1) at one or more positions selected from the 2^{nd} position and the 15^{th} position is not further mutated in (3).

The Fab region-binding peptide according to the present invention has superior binding capability to a Fab region of an immunoglobulin G (IgG).

The term “immunoglobulin” is a glycoprotein produced by a B cell of a lymphocyte and has a function to recognize a molecule such as a specific protein to be bound. An immunoglobulin has not only a function to specifically bind to a specific molecule, i.e. antigen, but also a function to detoxify and remove an antigen-containing factor in cooperation with other biological molecule or cell. An immunoglobulin is generally referred to as “antibody”, and the name is inspired by such functions. All of immunoglobulins basically have the same molecular structure. The basic structure of an immunoglobulin is a Y-shaped four-chain structure consisting of two light chains and two heavy chains of polypeptide chains. A light chain (L chain) is classified into two types of A chain and K chain, and all of immunoglobulins have either of the types. A heavy chain (H chain) is classified into five types of γ chain, μ chain, α chain, δ chain and ε chain, and an immunoglobulin is classified into isotypes depending on the kind of a heavy chain. An immunoglobulin G (IgG) is a monomer immunoglobulin, is composed of two heavy chains (γ chains) and two light chains, and has two antigen-binding sites.

A lower half vertical part in the “Y” shape of an immunoglobulin is referred to as a “Fc region”, and an upper half “V” shaped part is referred to as a “Fab region”. A Fc region has an effector function to initiate a reaction after binding of an antibody to an antigen, and a Fab region has a function to bind to an antigen. The Fab region and Fc region of a heavy chain are bound to each other through a hinge part. Papain, which is a proteolytic enzyme and which is contained in papaya, decomposes a hinge part to cut into two Fab regions and one Fc region. The part close to the end of the “Y” shape in a Fab region is referred to as a “variable region (V region)”, since there are various changes in the amino acid sequence in order to bind to various antigens. A variable region of a light chain is referred to as a “VL region”, and a variable region of a heavy chain is referred to as a “VH region”. The other part in a Fab region except for a V region and a Fc region are referred to as a “constant region (C region)”, since there is relatively less change. The constant region of a light chain is referred to as a “CL region”, and the constant region of a heavy chain is referred to as a “CH region”. A CH region is further classified into three regions of CH1 to CH3. A Fab region of a heavy chain is composed of a VH region and CH1, and a Fc region of a heavy chain is composed of CH2 and CH3. There is a hinge part between CH1 and CH2. More specifically, SpG-β binds to a CH1 region (CH1γ) and a CL region of IgG, and particularly to a CH1 region mainly (Non-patent Document 5).

The Fab region-binding peptide according to the present invention binds to a Fab region of an immunoglobulin G. A protein to which the present invention peptide binds may be an immunoglobulin G molecule containing both of a Fab region and a Fc region or a derivative of an immunoglobulin G molecule as long as the protein contains a Fab region. Such an immunoglobulin G molecule derivative to be bound by the Fab region-binding peptide of the present invention is not particularly restricted as long as the derivative contains a Fab region. For example, the derivative is exemplified by a Fab fragment which is fragmented to only a Fab region of an immunoglobulin G, a chimera immunoglobulin G prepared by replacing a part of domains of an immunoglobulin G with a domain of an immunoglobulin G derived from other organism to be fused, an immunoglobulin G of which a sugar chain in a Fc region is subjected to molecular alteration, and a Fab fragment to which a drug covalently binds.

In the present invention, the term “peptide” means any molecules having a polypeptide structure. In the range of the “peptide”, not only a so-called protein but also a fragmented peptide and a peptide to which other peptide is bound through a peptide bond are included.

Protein G (SpG) is a protein derived from a cell wall of Streptococcus sp. classified into Group G. Protein G has a capability to bind to an immunoglobulin G (IgG) of most mammals, strongly binds to a Fc region of IgG, and also weakly binds to a Fab region of IgG.

A SpG functional domain having a IgG-binding capability is referred to as “β domain, i.e. SpG-β”. The domain is referred to as β (B) domain or C domain (refer to Akerstrom et al., J. Biol. Chem., 1987, 28, p. 13388-, FIG. 5), but is referred to as “β domain” in the present invention in accordance with the definition of Fahnestock et al. (Fahnestock et al., J. Bacteriol., 1986, 167, p. 870-). The details of the amino acid sequence of SpG-β are different depending on the kind and strain of a bacterium from which the SpG-β is derived. As the typical amino acid sequences, with respect to two β domains, β1 and β2, derived from Group G Streptococcus sp. GX7809 strain, the amino acid sequence of β1 domain (SpG-β1) of which Asp at the 1^{st} position is substituted by Thr is shown as SEQ ID NO: 1, and the amino acid sequence of β2 domain (SpG-β2) is shown as SEQ ID NO: 2. Each β domain of SpG is collectively referred to Protein G-β domain (SpG-β), since the amino acid sequences of each β domain of SpG have high sequence identity with each other. The above-described substitution in SpG-β1 of SEQ ID NO: 1 has no effect on the affinity of the peptide having the amino acid sequence of SEQ ID NO: 1 for a Fab region. For example, the 1^{st} position in SpG-β2 derived from the GX7809 strain is Thr, and the amino acid sequences on and after the 2^{nd} position of SEQ ID NO: 1 and SEQ ID NO: 2 are described as amino acid sequences of each domain of SpG in some documents. From the standpoint, in the Fab region-binding peptide (1), the amino acid sequence derived from SpG-β1, SpG-β2 and SpG-β2 variant means the amino acid sequence of each domain itself or a variant amino acid which is mutated to the extent that the affinity of each domain for IgG-Fab is maintained.

Alexander et al. found that the denaturation midpoint temperatures of SpG-β1 and SpG-β2 at pH 5.4 are respectively 87.5° C. and 79.4° C. (Alexander et al., Biochemistry, 1992, 31, p. 3597-). Accordingly, as one of preferred embodiments of the present invention, a mutation is introduced into SpG-β1 (SEQ ID NO: 1) in terms of thermal stability of a peptide. In addition, as a SpG-β2 variant having excellent thermal stability, for example, an amino acid sequence of SEQ ID NO: 3 described in JP 2003-88381 A is exemplified. The amino acid sequence corresponds to the amino acid sequence of C36 peptide having SEQ ID NO: 8 described in JP 2003-88381 A, and Met for expression in Escherichia coli is added to the N-terminal of the sequence and Cys for immobilization on a water-insoluble carrier is added to the C-terminal of the sequence.

The term “domain” means a unit of higher-order structure of a protein. For example, a domain is composed of from dozens to hundreds of amino acid residues, and means a protein unit which can sufficiently serve some kind of a physicochemical or biochemical function.

The term “variant” means a protein or peptide obtained by introducing at least one substitution, addition and/or deletion into a sequence of a wild protein or peptide.

The present invention relates to a peptide having a sequence obtained by introducing the mutation into wild SpG-β. As the amino acid sequence before introducing the mutation, an amino acid sequence except for the sequences of wild SpG-β1 (SEQ ID NO: 1), wild SpG-β2 (SEQ ID NO: 2) and SpG-β2 variant (SEQ ID NO: 3) may be also used as long as a finally obtained peptide from the sequence is included in the range of the present invention. For example, even when the second domain (SEQ ID NO: 4) from the N-terminal side among three IgG-binding domains contained in SpG derived from Streptococcus sp. G148 strain or GX7805 strain classified in Group G is used, a similar effect can be obviously obtained. The domain has a sequence obtained by substituting the Ile at the 6^{th} position and Leu at the 7^{th} position of the amino acid sequence (SEQ ID NO: 1) of wild SpG β-1 with Val and Ile respectively. The present invention can be obviously applied to the domain, since the domain achieves a similar effect. Many of Protein G variants having excellent thermal stability are known, and are exemplified by those having the amino acid sequences described in WO 1997/026930 and JP 2003-88381 A.

In the present invention, a mutation to substitute an amino acid is described by adding a wild or non-mutated amino acid residue before the number of substituted position and adding a mutated amino acid residue after the number of substituted position. For example, the mutation to substitute Gly at 29^{th} position by Ala is described as G29A.

According to the present invention, a variant of which binding capability to a Fab region of an immunoglobulin G is superior to that before introducing the mutation of amino acid substitution is obtained by introducing the mutation of amino acid substitution to the specific position of β1 domain (SEQ ID NO: 1) or β2 domain (SEQ ID NO: 2) of wild SpG or β2 domain variant (SEQ ID NO: 3). In other words, the Fab region-binding peptide according to the present invention is characterized in having the binding capability to a Fab region of an immunoglobulin G superior to that before introducing the mutation of amino acid substitution. The phrase “before introducing the mutation” as a comparative control means before introducing the mutation of amino acid substitution according to the present invention, and does not mean before the mutation of SEQ ID NO: 3. The mutation of SEQ ID NO: 3 is introduced into β2 domain of wild SpG for thermal stability as described above. Specifically, the phrase means before introducing the mutation at the 2^{nd} position and/or the 15^{th} position of the amino acid sequences of SEQ ID NOs: 1 to 3.

For example, the affinity for a Fab region of immunoglobulin G can be evaluated by using a biosensor such as Biacore system (manufactured by GE Healthcare Bioscience) utilizing surface plasmon resonance principle; however, the method is not restricted thereto.

With respect to a condition for measuring a binding capability to a Fab region, a binding signal at the time of binding to a Fab region of an immunoglobulin G may be detected. For example, the binding capability can be easily measured at a constant temperature of 20 to 40° C. and in a neutral condition of pH 6 to 8.

An immunoglobulin G molecule as a binding partner is not restricted as long as a binding to a Fab region thereof can be detected. However, it is preferred to use a fragmented and purified Fab region, since a binding to a Fc region is also detected in the case of using an immunoglobulin G containing a Fc region. The difference of affinity can be easily evaluated by obtaining bonding response curves to the same immunoglobulin G molecule in the same measurement condition, analyzing the curves to obtain binding parameters, and comparing the parameters between a proteins before introducing the mutation and after introducing the mutation.

For example, as a binding parameter, an association constant (K_{A}) and a dissociation constant (K_{D}) can be used (Nagata et al., “Real-Time Analysis Experimental Method for Interaction Between Biological Substances”, Springer-Verlag Tokyo, 1998, p. 41). An association constant between the present invention variant and a Fab fragment can be measured by immobilizing a Fab fragment on a sensor tip and adding the present invention variant into a flow channel under a condition of the temperature of 25° C. and pH 7.4 in Biacore system. As the peptide having a sequence into which the mutation according to the present invention is introduced, a peptide of which association constant (K_{A}) is improved 2 or more times in comparison with a peptide before introducing the mutation is preferably used. The improvement rate of the preferably used peptide is more preferably 3 times or more, even more preferably 4 times or more, and even more preferably 5 times or more and 50 times or less.

The Fab region-binding peptide (1) according to the present invention has the amino acid sequence of SEQ ID NO: 1 corresponding to an amino acid sequence derived from β1 domain of protein G, an amino acid sequence of SEQ ID NO: 2 derived from β2 domain of protein G or an amino acid sequence of SEQ ID NO: 3 derived from β2 domain variant of protein G which have one or more amino acid substitutions at the position selected from the 2^{nd} position and the 15^{th} position. In the amino acid sequences of SEQ ID NOs: 1 to 3, the amino acid residue at the 2^{nd} position is Thr and the amino acid residue at the 15^{th} position is Glu. With respect to the substitution mutation according to the present invention, the amino acid residue at the 2^{nd} position is preferably substituted by Arg, and the amino acid residue at the 15^{th} position is preferably substituted by Tyr or Trp and more preferably Tyr.

The range of “one or more” in the phrase “amino acid sequence with deletion, substitution and/or addition of one or more amino acid residues” of the Fab region-binding peptide (2) according to the present invention is not particularly restricted as long as the Fab region-binding peptide with deletion or the like has a strong binding force to a Fab region of an immunoglobulin G. The above-described range of “one or more” is exemplified by 1 or more and 30 or less, preferably 1 or more and 20 or less, more preferably 1 or more and 10 or less, even more preferably 1 or more and 7 or less, even more preferably 1 or more and 5 or less, and particularly preferably 1 or more and 3 or less, 1 or 2, or 1.

The position at which one or more amino acids are substituted may be any positions except for the above-described 2^{nd} position and 15^{th} position. For example, the position to be substituted is preferably the 6^{th} position, the 7^{th} position, the 19^{th} position, the 24^{th} position, the 28^{th} position, the 29^{th} position, the 31^{st} position, the 35^{th} position, the 40^{th} position, the 42^{nd} position and the 47^{th} position. The kinds of amino acids at the preferred position are different from each other among SEQ ID NOs: 1 to 3. In addition, in terms of a steric structure, the position to be substituted is preferably the 10^{th} position, the 13^{th} position, the 18^{th} position, the 19^{th} position, the 21^{st} position, the 28^{th} position, the 30^{th} position, the 31^{st} position, the 33^{rd} position and the 39^{th} position. The position at which one or more amino acids are deleted or added is basically preferably the N-terminal and C-terminal.

Even when the number of amino acids is changed by the above-described deletion or addition, the position of an amino acid residue after introducing the mutation which corresponds to the position of an amino acid residue before introducing the mutation can be easily identified by alignment analysis between the amino acid sequences before and after introducing the mutation. The means of such an alignment analysis is widely known by a person skilled in the art.

With respect to the Fab region-binding peptide (3) according to the present invention, the sequence identity is preferably 80% or more, more preferably 85% or more, even more preferably 90% or more, and particularly preferably 95% or more. The sequence identity can be measured by a program for amino acid sequence multiple alignment, such as Clustal (http://www.clustal.org/omega/). Even when the number of amino acids in the amino acid sequence before introducing the mutation is different, a person skilled in the art can easily identify the position corresponding to the 2^{nd} position and the 15^{th} position in SEQ ID NO: 1 in the case where the sequence identity is 80% or more.

The Fab region-binding peptide (2) or Fab region-binding peptide (3) according to the present invention is prepared by further introducing a mutation into the Fab region-binding peptide (1) in addition to the already-introduced mutation of amino acid substitution, and has higher binding capability than the domain having the amino acid sequence of SEQ ID NOs: 1 to 3, which corresponds to the amino acid sequence of the Fab region-binding peptide (1) before introducing the mutation of amino acid substitution.

Protein G is a protein which contains 2 or 3 immunoglobulin-binding domains in the form of tandem line. As one of the embodiments, the Fab region-binding peptide according to the present invention may be a multimer of 2 or more monomers or single domains of the Fab region-binding peptide connected each other. The number of the monomers or single domains is preferably 3 or more, more preferably 4 or more, and even more preferably 5 or more. The upper limit of the number of connected domains may be, for example, 10, preferably 8, and more preferably 6. Such a multimer may be a homomultimer in which one kind of Fab region-binding peptides are connected, such as homodimer and homotrimer, or a heteromultimer in which two or more kinds of Fab region-binding peptides are connected, such as heterodimer and heterotrimer.

A method for connecting monomer proteins according to the present invention is exemplified by a connecting method through one or more amino acid residues and a method for directly connecting the monomer proteins without using an amino acid residue; however, is not restricted to the exemplified methods. The number of the amino acid residue for connection is not particularly restricted, and is preferably 1 residue or more and 20 residues or less, more preferably 15 residues or less, even more preferably 10 residues or less, even more preferably 5 residues or less, and even more preferably 2 residues or less. It is preferred to use a sequence which connects β1 and β2 or β2 and β3 of wild SpG. From another point of view, it is preferred that the amino acid residue for connection does not destabilize a three dimensional structure of the monomer protein.

The Fab region-binding peptide according to the present invention may contain any one of the amino acid sequences of the Fab region-binding peptides (1) to (3), and other peptide and compound may be bound thereto. In other words, the amino acid sequence of the Fab region-binding peptide according to the present invention may be any one of the amino acid sequences of SEQ ID NOs: 1 to 3 with mutation at the 2nd position and/or the 15^{th} position and may contain other amino acid sequence or other compound to be bound. For example, as one embodiment, a fusion peptide prepared by fusing the Fab region-binding peptide according to the present invention as one structural component or the peptide multimer of the two or more peptides with other peptide having different function is exemplified. Such a fusion peptide is exemplified by a peptide fused with albumin or GST, i.e. glutathione S-transferase, but is not restricted to the examples. In addition, peptides fused with a nucleic acid such as DNA aptamer, a drug such as antibiotic or a polymer such as PEG, i.e. polyethylene glycol, are also included in the range of the present invention as long as such a fusion peptide utilizes the utility of the present invention peptide. However, it is preferred that the amino acid sequence of the Fab region-binding peptide according to the present invention is composed of any one of amino acid sequences of SEQ ID NOs: 1 to 3. Even in such a case, the Fab region-binding peptide according to the present invention may be immobilized on a water-insoluble carrier through a linker group as described later, and the Fab region-binding peptides may be connected each other through a linker group in the case of a multimer.

It is also included in the range of the present invention as one embodiment to use the above-described present invention peptide as an affinity ligand having an affinity for an immunoglobulin or a fragment thereof, particularly having an affinity for a Fab region. An affinity separation matrix prepared by immobilizing the ligand on a water-insoluble carrier is similarly included in the range of the present invention as one embodiment. The term “affinity ligand” in the disclosure means a substance and a functional group to selectively bind to and adsorb a target molecule from an aggregate of molecules on the basis of a specific affinity between molecules, such as binding between an antigen and an antibody, and means the peptide which specifically binds to an immunoglobulin G in the present invention. In the present invention, the term “ligand” also means an “affinity ligand”.

The water-insoluble carrier usable in the present invention is exemplified by an inorganic carrier such as glass beads and silica gel; an organic carrier; and a composite carrier obtained from the combination of the above carriers, such as an organic-organic composite carrier and an organic-inorganic composite carrier. An organic carrier is exemplified by a synthetic polymer carrier such as cross-linked polyvinyl alcohol, cross-linked polyacrylate, cross-linked polyacrylamide and cross-linked polystyrene; a polysaccharide carrier such as crystalline cellulose, cross-linked cellulose, cross-linked agarose and cross-linked dextran. The commercial product thereof is exemplified by porous cellulose gel GCL2000, Sephacryl (registered trademark) S-1000 prepared by crosslinking allyl dextran and methylene bisacrylamide through a covalent bond, an acrylate carrier Toyopearl (registered trademark), a cross-linked agarose carrier Sepharose (registered trademark) CL4B, and a cross-linked cellulose carrier Cellufine (registered trademark). However, it should be noted that the water-insoluble carrier usable in the present invention is not restricted to the carriers exemplified as the above.

It is preferred that the water-insoluble carrier usable in the present invention has large surface area and that the carrier is porous with a large number of fine pores having a suitable size in terms of a purpose and method for using the affinity separation matrix according to the present invention. The carrier can have any form such as beads, monolith, fiber and film (including hollow fiber), and any form can be selected.

With respect to a method for immobilizing the ligand, for example, the ligand can be bound to a carrier by a conventional coupling method utilizing an amino group, a carboxy group or a thiol group of the ligand. Such a coupling method is exemplified by an immobilization method including activation of a carrier by a reaction with cyanogen bromide, epichlorohydrin, diglycidyl ether, tosyl chloride, tresyl chloride, hydrazine, sodium periodate or the like, or introduction of a reactive functional group on the carrier surface, and the coupling reaction between the resulting carrier and a compound to be immobilized as a ligand; and an immobilization method by condensation and crosslinking which method includes adding a condensation reagent such as carbodiimide or a reagent having a plurality of functional groups in the molecule, such as glutaraldehyde, into a mixture containing a carrier and a compound to be immobilized as a ligand.

A spacer molecule composed of a plurality of atoms may be introduced between the ligand and carrier. Alternatively, the ligand may be directly immobilized on the carrier. Accordingly, the Fab region-binding peptide according to the present invention may be chemically modified for immobilization, or may have an additional peptide containing 1 or more and 100 or less amino acid residues useful for immobilization as a linker group. Such an amino acid useful for immobilization is exemplified by an amino acid having a functional group useful for a chemical reaction for immobilization in a side chain, and specifically exemplified by Lys having an amino group in a side chain and Cys having a thiol group in a side chain. The number of the amino acid residue contained in the above-described peptide linker group is preferably 50 or less, more preferably 40 or less or 20 or less, and even more preferably 10 or less. Since the binding capability of the peptide according to the present invention to a Fab region is principally maintained in a matrix prepared by immobilizing the peptide as a ligand in the present invention, any modification and change for immobilization are included in the range of the present invention.

It becomes possible by using the affinity separation matrix of the present invention that a peptide containing a Fab region of an immunoglobulin G is purified in accordance with affinity column chromatography purification method. A peptide containing a Fab region of an immunoglobulin G can be purified by a procedure in accordance with a method for purifying an immunoglobulin by affinity column chromatography, for example, by a method using SpA affinity separation matrix (Non-Patent Document 1). Specifically, after a buffer which contains a peptide containing a Fab region of an immunoglobulin G and of which pH is approximately neutral is prepared, the solution is allowed to pass through an affinity column filled with the affinity separation matrix of the present invention so that the peptide containing a Fab region of an immunoglobulin G is adsorbed. Then, an appropriate amount of a pure buffer is allowed to pass through the affinity column to wash the inside of the column. At the time, the target peptide containing a Fab region of an immunoglobulin G is still adsorbed on the affinity separation matrix of the present invention in the column. The affinity separation matrix on which the peptide according to the present invention is immobilized as a ligand is excellent in the absorption and retention performance of a target peptide containing a Fab region of an immunoglobulin G from the step of adding a sample through the step of washing the matrix. Then, an acid buffer of which pH is appropriately adjusted is allowed to pass through the column to elute the target peptide containing a Fab region of an immunoglobulin G. As a result, purification with high purity can be achieved. Into the acid buffer for elution, a substance for promoting dissociation from the matrix may be added.

The affinity separation matrix according to the present invention can be reused by allowing an adequate strong acid or strong alkali pure buffer which does not completely impair the function of the ligand compound or the base material of the carrier to pass through the matrix for wash. As the wash solution, a solution containing an adequate modifying agent or an organic solvent may be used.

The present invention also relates to a DNA which encodes the peptide of the present invention. The DNA encoding the present invention peptide may be any DNA as long as the amino acid sequence produced from translation of the base sequence of the DNA constitutes the present invention peptide. Such a base sequence can be obtained by a generally used known technologies, for example, by polymerase chain reaction (hereinafter, abbreviated as “PCR”) method. Alternatively, the base sequence can be synthesized by publicly-known chemical synthesis method or is available from DNA library. A codon in the base sequence may be substituted by a degenerate codon, and the base sequence is not necessarily the same as the original base sequence as long as the translated amino acids are the same as those encoded by the original base sequence. It is possible to obtain a recombinant DNA having the one or more base sequences, a vector containing the recombinant DNA, such as a plasmid and a phage, a transformant transformed by the vector having the DNA, a genetically engineered organism having the DNA introduced therein, or a cell-free protein synthesis system using the DNA as a template for transcription.

The Fab region-binding peptide according to the present invention may be obtained as a fusion peptide fused with a publicly-known protein which beneficially has an action to assist the expression of a protein or to facilitate the purification of a protein. In other words, it is possible to obtain a microorganism or cell containing at least one recombinant DNA encoding a fusion peptide containing the Fab region-binding peptide according to the present invention. The above-described protein is exemplified by a maltose-binding protein (MBP) and a glutathione S-transferase (GST), but is not restricted thereto.

Site-specific mutagenesis for modifying the DNA encoding the peptide of the present invention can be carried out using recombinant DNA technology, PCR method or the like as follows.

Specifically, mutagenesis by recombinant DNA technology can be carried out as follows: for example, in the case where there are suitable restriction enzyme recognition sequences on both sides of a target mutagenesis site in the gene encoding the present invention peptide, cassette mutagenesis method can be carried out in which method a region containing the target mutagenesis site is removed by cleaving the restriction enzyme recognition sites with the above-described restriction enzymes and then a mutated DNA fragment is inserted. Into the mutated DNA fragment, mutation is introduced only at the target site by a method such as chemical synthesis.

For example, site-directed mutagenesis by PCR can be carried out by double primer mutagenesis. In double primer mutagenesis, PCR is carried out by using a double-stranded plasmid encoding the present invention peptide as a template, and using two kinds of synthesized oligo primers which contain complementary mutations in the + strand and − strand.

A DNA encoding a multimer peptide can be produced by ligating the desired number of DNAs each encoding the monomer peptide (single domain) of the present invention to one another in tandem. For example, with respect to connecting method for the DNA encoding the multimer peptide, a suitable restriction enzyme site is introduced in the DNA sequence and double-stranded DNA fragments cleaved with the restriction enzyme are ligated using a DNA ligase. One restriction enzyme site may be introduced or a plurality of restriction enzyme sites of different types may be introduced. When the base sequences encoding each monomer peptide in the DNA encoding the multimer peptide are the same, homologous recombination may be possibly induced in a host. Thus, the sequence identity between base sequences of DNAs encoding the monomer peptides to be ligated may be 90% or less, preferably 85% or less, more preferably 80% or less, and even more preferably 75% or less. The identity of the base sequence can be also determined by an ordinary method similarly to the amino acid sequence.

The “expression vector” of the present invention contains a base sequence encoding the above-described peptide of the present invention or a part of the amino acid sequence of the peptide, and a promoter which can be operably linked to the base sequence to function in a host. In general, the expression vector can be obtained by linking or inserting the gene encoding the present invention peptide to a suitable vector. The vector into which the gene is inserted is not particularly restricted as long as the vector is capable of autonomous replication in a host. As such a vector, a plasmid DNA or a phage DNA can be used. For example, in the case of using Escherichia coli as a host, a pQE series vector (manufactured by QIAGEN), a pET series vector (manufactured by Merck), a pGEX series vector (manufactured by GE Healthcare Bioscience) or the like can be used.

The transformant of the present invention can be produced by introducing the recombinant vector of the present invention into a host cell. A method for introducing the recombinant DNA into a host is exemplified by a method using a calcium ion, electroporation method, spheroplast method, lithium acetate method, agrobacterium infection method, particle gun method and polyethylene-glycol method, but is not restricted thereto. A method for expressing the function of the obtained gene in a host is also exemplified by a method in which the gene obtained by the present invention is implanted into a genome (chromosome). A host cell is not particularly restricted, and bacteria (eubacteria) such as Escherichia coli, Bacillus subtilis, Brevibacillus, Staphylococcus, Streptococcus, Streptomyces and Corynebacterium can be preferably used in terms of mass production in a low cost.

The Fab region-binding peptide according to the present invention can be produced by culturing the above-described transformant in a medium to allow the cell to produce and accumulate the peptide of the present invention in the cultured bacterial cell (including the periplasmic space of the bacterial cell) or in the culture liquid (outside the bacterial cell), and collecting the desired peptide from the culture. In addition, the peptide of the present invention can also be produced by culturing the above-described transformant in a medium to allow the cell to produce and accumulate a fusion protein containing the peptide of the present invention in the cultured bacterial cell (including the periplasmic space of the cell) or in the culture liquid (outside the cell), collecting the fusion peptide from the culture, cleaving the fusion peptide with a suitable protease, and collecting the desired peptide.

The transformant of the present invention can be cultured in a medium in accordance with a common method for culturing a host cell. The medium used for culturing the obtained transformant is not particularly restricted as long as the medium enables high yield production of the present invention peptide with high efficiency. Specifically, carbon source and nitrogen source, such as glucose, sucrose, glycerol, polypeptone, meat extract, yeast extract and casamino acid, can be used. In addition, an inorganic salt such as potassium salt, sodium salt, phosphate, magnesium salt, manganese salt, zinc salt and iron salt is added as required. In the case of an auxotrophic host cell, a nutritional substance necessary for the growth thereof may be added. In addition, an antibiotic such as penicillin, erythromycin, chloramphenicol and neomycin may be added as required.

Furthermore, in order to inhibit the degradation of the target peptide caused by a host-derived protease present inside or outside the bacterial cell, a publicly-known protease inhibitor and/or other commercially available protease inhibitor may be added in an appropriate concentration. The publicly-known protease inhibitor is exemplified by phenylmethane sulfonyl fluoride (PMSF), benzamidine, 4-(2-aminoethyl)-benzenesulfonyl fluoride (AEBSF), antipain, chymostatin, leupeptin, Pepstatin A, phosphoramidon, aprotinin and ethylenediaminetetraacetic acid (EDTA).

In order to obtain rightly folded Fab region-binding peptide according to the present invention, for example, a molecular chaperone such as GroEL/ES, Hsp70/DnaK, Hsp90 and Hsp104/ClpB may be used. For example, such a molecular chaperone is co-existed with the present invention peptide by coexpression or as a fusion protein. As a method for obtaining rightly folded present invention peptide, addition of an additive for assisting right folding into the medium and culturing at a low temperature are exemplified, but the method is not restricted thereto.

The medium for culturing transformant produced from an Escherichia coli as a host is exemplified by LB medium containing triptone 1%, yeast extract 0.5% and NaCl 1%, 2×YT medium containing triptone 1.6%, yeast extract 1.0% and NaCl 0.5%, or the like.

For example, the transformant may be aerobically cultured in an aeration-stirring condition at a temperature of 15 to 42° C., preferably 20 to 37° C., for several hours to several days. As a result, the peptide of the present invention is accumulated in the cultured cell (including the periplasmic space of the cell) or in the culture liquid (outside the cell) to recover the peptide. In some cases, the culturing may be performed anaerobically without aeration. In the case where a recombinant peptide is secreted, the produced recombinant peptide can be recovered after the culture period by separating the supernatant containing the secreted peptide using a common separation method such as centrifugation and filtration from the cultured cell. In addition, in the case where the peptide is accumulated in the cultured cell (including the periplasmic space), the peptide accumulated in the cell can be recovered, for example, by collecting the bacterial cell from the culture liquid by centrifugation, filtration or the like, and then disrupting the bacterial cell by sonication, French press method or the like, and/or solubilizing the bacterial cell by adding a surfactant or the like.

A method for purifying the peptide of the present invention can be carried out by any one or an appropriate combination of techniques such as affinity chromatography, cation or anion exchange chromatography, gel filtration chromatography or the like. It can be confirmed whether the obtained purified substance is the target peptide or not by an ordinary method such as SDS polyacrylamide gel electrophoresis, N-terminal amino acid sequence analysis and Western blot analysis.

The present application claims the benefit of the priority date of Japanese patent application No. 2014-174074 filed on Aug. 28, 2014. All of the contents of the Japanese patent application No. 2014-174074 filed on Aug. 28, 2014, are incorporated by reference herein.

### EXAMPLES

Hereinafter, the present invention is described in more detail with Examples. However, the present invention is not restricted to the following Examples.

The peptide variant obtained in the following Examples is described as “domain-introduced mutation”, and wild type into which mutation is not introduced is described as “domain-Wild”. For example, β1 domain of wild SpG having SEQ ID NO: 1 is described as “GB1-Wild”, and SpG-β1 domain variant into which mutation to substitute Thr at the 2^{nd} position by Arg (the mutation is described as “T02R”) is described as “GB1-T02R”. The amino acid sequence of SEQ ID NO: 3 of β2 domain variant having excellent thermal stability is described as “GB2*-Wild” in the present disclosure, since the amino acid sequence is regarded as an amino acid sequence before introducing the mutation of the present invention.

With respect to a variant having two kinds of mutations, the mutations are described together with a slash. For example, SpG β1 domain variant into which mutations of T02R and E15Y are introduced is described as “GB1-T02R/E15Y”.

With respect to a protein in which a plurality of single domains are connected, the number of the connected domains and “d” are added after a period. For example, a protein in which two SpG β1 domain variants having mutations of T02R and E15Y are connected is described as “GB1-T02R/E15Y.2d”.

When a Cys residue, i.e. “C”, having a functional group for immobilization is added to the C-terminal in order to immobilize a protein on a water-insoluble carrier, one letter code of the added amino acid is added after “d”. For example, a protein in which two SpG β1 domain variants having mutations of T02R and E15Y are connected and to which Cys is added at the C-terminal is described as “GB1-T02R/E15Y.2dC”.

### Example 1: Preparation of SpG Variants

(1) Preparation of Expression Plasmids of SpG Variants

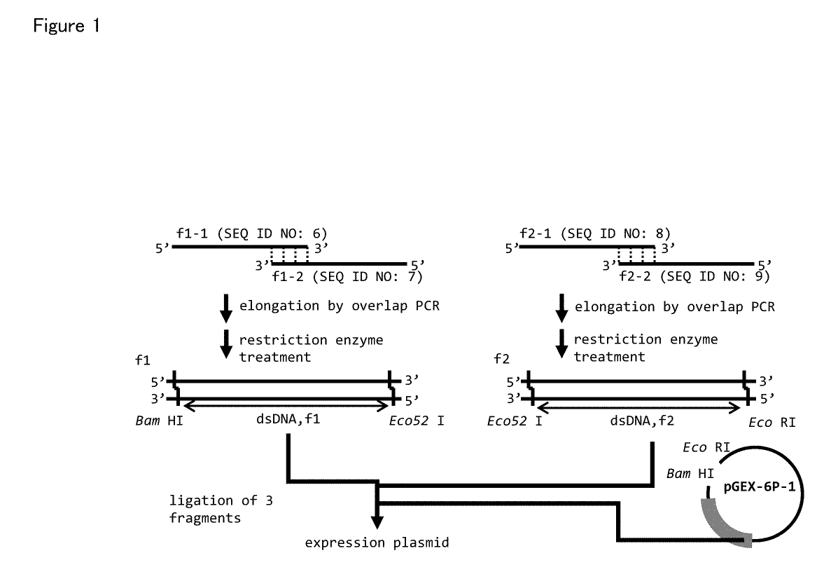

The amino acid sequence of SEQ ID NO: 3 of SpG β2 domain variant having excellent thermal stability is a sequence of a comparative example before introducing the mutation of the present invention, and is expedientially described as “GB2*-Wild” in the present disclosure. A base sequence of SEQ ID NO: 5 encoding the peptide having the amino acid sequence of GB2*-Wild.1d (SEQ ID NO: 3) was designed by reverse translation from the amino acid sequence. The method for producing the expression plasmid is shown in FIG. 1. A DNA encoding GB2*-Wild.1d was prepared by ligating two kinds of double-stranded DNAs (f1 and f2) having the same restriction enzyme site, and integrated into the multiple cloning site of an expression vector. In fact, the preparation of the peptide-coding DNA and the integration into the vector were simultaneously performed by ligating three fragments for connecting three double-stranded DNAs of the two kinds of double-stranded DNAs and an expression vector. The two kinds of double-stranded DNAs were prepared by elongating two kinds of single-stranded DNAs (f1-1/f1-2 or f2-1/f2-2) respectively containing about 30-base complementary region with overlapping PCR. Hereinafter, the specific experimental procedure is described. Single-stranded oligo DNAs f1-1 (SEQ ID NO: 6)/f1-2 (SEQ ID NO: 7) were synthesized by outsourcing to Sigma Genosys. The overlapping PCR was performed using Blend Taq (manufactured by TOYOBO CO., LTD.) as a polymerase. The PCR product was subjected to agarose electrophoresis and the target band was cut out. The thus extracted double-stranded DNA was cleaved with the restriction enzymes BamHI and Eco52I (both available from Takara Bio, Inc.). Similarly, single-stranded oligo DNAs f2-1 (SEQ ID NO: 8)/f2-2 (SEQ ID NO: 9) were synthesized by outsourcing. The double-stranded DNA synthesized by overlapping PCR was extracted and cleaved with the restriction enzymes Eco52I and EcoRI (both available from Takara Bio, Inc.). Then, the two kinds of double-stranded DNAs were sub-cloned into the BamHI/EcoRI site in the multiple cloning site of the plasmid vector pGEX-6P-1 (GE Healthcare Bioscience). The ligation reaction for the subcloning was performed using Ligation high (manufactured by TOYOBO CO., LTD.) in accordance with the protocol attached to the product.

A competent cell (“Escherichia coli HB101” manufactured by Takara Bio, Inc.) was transformed using the above-described plasmid vector pGEX-6P-1 in accordance with the protocol attached to the competent cell product. By using the plasmid vector pGEX-6P-1, GB2*-Wild.1d which was fused with glutathione-S-transferase (hereinafter, abbreviated as “GST”) could be produced. Then, the plasmid DNA was amplified and extracted using a plasmid purification kit (“Wizard Plus SV Minipreps DNA Purification System” manufactured by Promega) in accordance with the standard protocol attached to the kit. The base sequence of the peptide-coding DNA of the expression plasmid was determined by using a DNA sequencer (“3130xl Genetic Analyzer” manufactured by Applied Biosystems). The sequencing PCR was performed by using a gene analysis kit (“BigDye Terminator v. 1.1 Cycle Sequencing Kit” manufactured by Applied Biosystems) and DNA primers for sequencing the plasmid vector pGEX-6P-1 (manufactured by GE Healthcare Bioscience) in accordance with the attached protocol. The sequencing product was purified by using a plasmid purification kit (“BigDye XTerminator Purification Kit” manufactured by Applied Biosystems) in accordance with the attached protocol and used for the base sequence analysis.

The expression vectors of GB1-Wild.1d (SEQ ID NO: 1), GB2-Wild.1d (SEQ ID NO: 2) and GC2-Wild.1d (SEQ ID NO: 4) were prepared using suitable single-stranded DNAs in a similar manner. The base sequences of the peptide-coding DNAs in each expression plasmid are shown as SEQ ID NOs: 10 to 12.

With respect to DNAs encoding each SpG-β2*1 residue variant, expression plasmids into which further mutation was introduced were obtained in accordance with QuikChange method by using the prepared expression plasmid of GB2*-Wild.1d as a template and two kinds of primers selected from oligonucleotide primers of SEQ ID NOs: 11 to 20. The QuikChange method was carried out using a DNA polymerase Pfu Turbo and methylated DNA (template DNA) cleavage enzyme Dpn I (both were available from Stratagene) in accordance with the protocol of Stratagene. The sequences of the oligonucleotide primers of SEQ ID NOs: 13 to 18, the combination of two kinds of primers used in the QuikChange method, and the relation between the primers and introduced mutations are shown in Table 1. The oligonucleotide primers were prepared by outsourcing to Sigma Genosys unless otherwise noted. The peptide-coding DNA sequences and amino acid sequences of the each prepared SpG-β2*1 residue variants are shown as SEQ ID NOs: 19 to 24, and the relations between the sequences are shown in Table 1. The transformation and analysis of base sequence were carried out similarly to the above manner.

TABLE 1
Combination of promers and introduced amino acid mutation

| Introduced | | Amino acid |
|---|---|---|
| mutation | | sequence |
| Sequence | Primer sequence | Coding DNA |
| name | (template is expression plasmid of GB2*-Wild) | sequence |
| T02R | SEQ ID NO: 13 5′GGATCCACCAGATACAAACTGG 3′ | SEQ ID NO: 19 |
| GB2*- | SEQ ID NO: 14 5′CCAGTTTGTATCTGGTGGATCC 3′ | SEQ ID NO: 20 |

T02R.ld

| | | |
|---|---|---|
| E15Y | SEQ ID NO: 15 | SEQ ID NO: 21 |
| | 5′GACCCTGAAAGGTTATACCACCACCAAAG 3′ | |
| GB2*- | SEQ ID NO: 16 | SEQ ID NO: 22 |
| E15Y.ld | 5′CTTTGGTGGTGGTATAACCTTTCAGGGTC 3′ | |
| E15W | SEQ ID NO: 17 | SEQ ID NO: 23 |
| | 5′GACCCTGAAAGGTTGGACCACCACCAAAG 3′ | |
| GB2*- | SEQ ID NO: 18 | SEQ ID NO: 24 |
| E15W.ld | 5′CTTTGGTGGTGGTCCAACCTTTCAGGGTC 3′ | |

With respect to GB2-E15W.1d (SEQ ID NP: 25) obtained by introducing a mutation into wild SpG-β2*1, an expression plasmid having the DNA sequence (SEQ ID NO: 26) which encoded GB2-E15W.1d was prepared using the expression vector of GB2-Wild.1d (of which peptide-coding DNA sequence is SEQ ID NO: 11) as a template and primers having SEQ ID NOs: 17 and 18. The transformation and analysis of base sequence were carried out similarly to the above manner.

With respect to GB2*-T02R/E15Y.1d (SEQ ID NO: 27) as a two residues variant of SpG-β2*1, an expression plasmid having the DNA sequence (SEQ ID NO: 28) which encoded GB2*-T02R/E15Y.1d was prepared by QuikChange method using the expression plasmid of GB2*-T02R/E15Y.1d (of which peptide-coding DNA sequence is SEQ ID NO: 19) as a template and primers having SEQ ID NOs: 15 and 16. The transformation and analysis of base sequence were carried out similarly to the above manner.

(2) Preparation of SpG Variant

The transformant produced by integrating each of the SpG-β2* variant gene obtained in the above-described (1) was cultured in 2×YT medium containing ampicillin at 37° C. overnight. The culture liquid was inoculated in 2×YT medium containing about a 100-fold amount of ampicillin and cultured at 37° C. for about 2 hours. Then, IPTG, i.e. isopropyl-1-thio-β-D-galactoside, was added so that the final concentration thereof became 0.1 mM, and the transformant was further cultured at 37° C. for 18 hours.

After the culture, the bacterial cell was collected by centrifugation and re-suspended in 5 mL of PBS buffer. The cell was broken by sonication and centrifuged to separate a supernatant fraction as a cell-free extract and an insoluble fraction. When a target gene is integrated into the multiple cloning site of pGEX-6P-1 vector, a fusion peptide having GST added to the N-terminal is produced. Each fraction was analyzed by SDS electrophoresis; as a result, a peptide band assumed to be induced by IPTG was detected at a position corresponding to a molecular weight of about 25,000 or more in the lanes of each of all the cell-free extracts obtained from all of cultured liquids of each transformant. The molecular weights were approximately similar but the positions of bands were different depending on the kind of variant.

The GST fusion peptide was roughly purified from each of the cell-free extract containing the GST fusion peptide by affinity chromatography using a GSTrap FF column (GE Healthcare Bioscience), which had an affinity for GST. Specifically, each of the cell-free extract was added to the GSTrap FF column and the column was washed with a standard buffer (20 mM NaH₂PO₄—Na₂HPO₄, 150 mM NaCl, pH 7.4). Then, the target GST fusion peptide was eluted by using an elution buffer (50 mM Tris-HCl, 20 mM Glutathione, pH 8.0). As the sample used for assay in the following Examples with fusing GST, a peptide solution obtained by concentrating the eluent with centrifugal filter unit Amicon (manufactured by Merck Millipore) and replacing the solvent with a standard buffer was used.

When a gene is integrated into a multiple cloning site of pGEX-6P-1 vector, an amino acid sequence by which GST can be cleaved using sequence-specific protease: PreScission Protease (manufactured by GE Healthcare Bioscience) is inserted between GST and a target protein. By using such PreScission Protease, GST was cleaved in accordance with the attached protocol. The target peptide was purified by gel filtration chromatography using a Superdex 75 10/300 GL column (manufactured by GE Healthcare Bioscience) from the GST-cleaved sample used for assay. Each of the reaction mixture was added to the Superdex 75 10/300 GL column equilibrated with a standard buffer, and the target protein therein was separated and purified from the cleaved GST and PreScission Protease. The above-described all of the peptide purification by chromatography using the column was performed by using AKTAprime plus system (manufactured by GE Healthcare Bioscience). In addition, after the cleavage of GST, the peptide produced in the present Example had the sequence of Gly-Pro-Leu-Gly-Ser derived from the vector pGEX-6P-1 at the N-terminal side.

### Example 2: Evaluation of Affinity of SpG Variant for IgG-Fab

(1) Preparation of Fab Fragment Derived from IgG (IgG-Fab)

A humanized monoclonal IgG product as a raw material was fragmented into a Fab fragment and a Fc fragment using papain, and only the Fab fragment was separated and purified. Hereinafter, a method for producing IgG-Fab derived from anti-HER2 monoclonal antibody (generic name: Trastuzumab) is described. In the present disclosure, when other IgG-Fab was used for evaluation, the fragment was basically prepared in a similar method.

Specifically, a humanized monoclonal IgG product (“HERCEPTIN” manufactured by CHUGAI PHARMACEUTICAL CO., LTD., in the case of anti-HER2 monoclonal antibody) was dissolved in a buffer for papain treatment (0.1 M AcOH—AcONa, 2 mM EDTA, 1 mM cysteine, pH 5.5), and agarose on which papain was immobilized (“Papain Agarose from papaya latex” manufactured by SIGMA) was added thereto. The mixture was incubated with stirring by a rotator at 37° C. for about 8 hours. The IgG-Fab was purified by recovering the IgG-Fab as a flow-through fraction in an affinity chromatography using KanCapA column (manufactured by GE Healthcare Bioscience) from the reaction mixture which contained both of a Fab fragment and a Fc fragment and which was separated from the agarose on which papain was immobilized. The obtained crude IgG-Fab solution was subjected to purification by gel filtration chromatography using Superdex 75 10/300 GL column to obtain IgG-Fab solution. In the chromatography, a standard buffer was used for equilibration and separation. Similarly to the above-described Example 1, AKTAprime plus system was used in the chromatography for protein purification.

(2) Analysis of Affinity of SpG-β2*1 Amino Acid Residue Variant for IgG-Fab

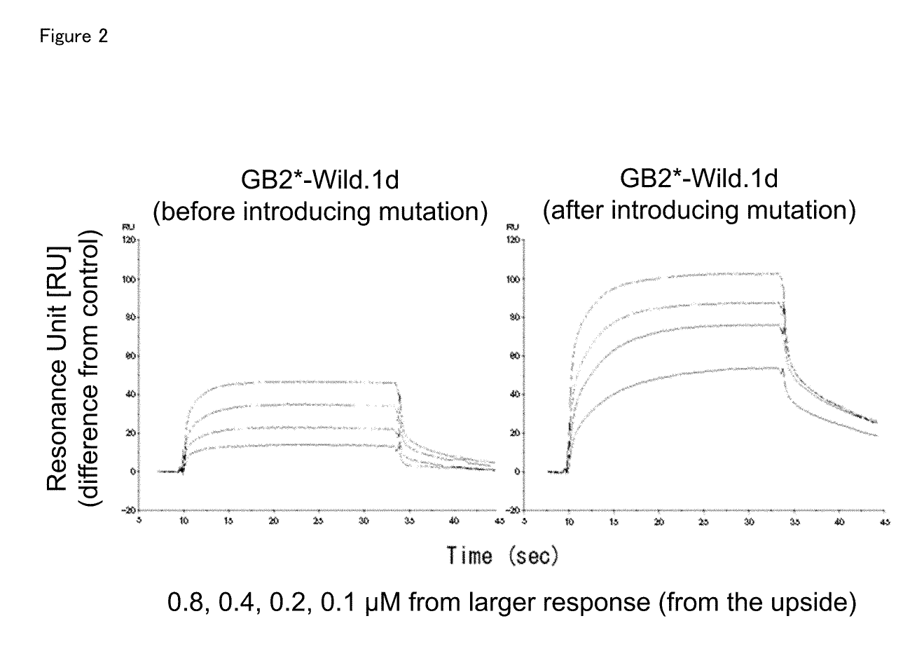

The affinity of each of the SpG-β2* variant obtained in the above-described Example 1(2) for the IgG-Fab of anti-HER2 monoclonal antibody was evaluated using a biosensor Biacore 3000 (manufactured by GE Healthcare Bioscience) utilizing surface plasmon resonance. In the present Example, the IgG-Fab obtained in the above-described Example 2(1) was immobilized on a sensor tip, and each of the peptide was flown on the tip to detect the interaction between the two. The IgG-Fab was immobilized on a sensor tip CM5 by amine coupling method using N-hydroxysuccinimide (NHS) and N-ethyl-N′-(3-dimethylaminopropyl)carbodiimide hydrochloride (EDC), and ethanolamine was used for blocking. All of the sensor tip and reagents for immobilization was manufactured by GE Healthcare Bioscience. The IgG-Fab solution was diluted to about 10 times using a buffer for immobilization (10 mM AcOH—AcONa, pH 4.5), and the IgG-Fab was immobilized on the sensor tip in accordance with the protocol attached to the Biacore 3000. In addition, a reference cell as negative control was also prepared by activating another flow cell on the tip with EDC/NHS and then immobilizing human serum albumin (manufactured by Wako Pure Chemical Industries, Ltd.). Peptide solutions of each of the SpG-β2* variant having concentrations of 0.1 μm, 0.2 μm, 0.4 μm and 0.8 μm were prepared using a running buffer (20 mM NaH₂PO₄—Na₂HPO₄, 150 mM NaCl, 0.005% P-20, pH 7.4). The peptide solution was added to the sensor tip in a flow rate of 10 μL/min for 4 minutes. Bonding response curves at the time of addition (association phase, for 4 minutes) and after the addition (dissociation phase, for 4 minutes) were sequentially obtained at a measurement temperature of 25° C. After each measurement, the added peptide remaining on the sensor tip was removed by flowing the running buffer for 5 minutes or more; and about 10 mM NaOH was added for wash between the measurements if required. The bonding response curve obtained by subtracting the bonding response curve of the reference cell was subjected to fitting analysis by a binding model of 1:1 using a software BIA evaluation attached to the system, and affinity constant (K_{A}=kₒₙ/k_{off}) to human IgG-Fab was calculated. The experimental data was shown in FIG. 2 and the analysis result is shown in Table 2.

TABLE 2

| | | | Anti-HER2 antibody - Fab | | | |
|---|---|---|---|---|---|---|
| | Sequence number | | kₒₙ | k_{off} | K_{A} | |
| SpG-β2* | Amino acid | Base | ×10⁴ | ×10⁻² | ×10⁶ | Vs. |
| variant | sequence | sequence | M⁻¹s | s⁻¹ | M⁻¹ | Wild |
| GB2*-Wild.1d | 3 | 5 | 8.8 | 3.8 | 2.3 | — |
| GB2*-T02R.1d | 19 | 20 | 9.8 | 1.0 | 9.8 | 4.3 |
| GB2*-E15Y.1d | 21 | 22 | 9.2 | 1.0 | 9.3 | 4.0 |
| GB2*-E15W.1d | 23 | 24 | 11.9 | 1.1 | 10.8 | 4.8 |

As the result shown in Table 2, it was confirmed that the association constant of the variant according to the present invention to IgG-Fab is improved, in other words, the binding force to IgG-Fab becomes stronger, in comparison with the peptide before introducing the mutation. It was found from the analysis result that the association constants of GB2*-T02R.1d, GB2*-E15Y.1d and GB2*-E15W.1d are 4 to 5 times larger than that of GB2*-Wild.1d before introducing the mutation.

As an important point, the above-described GB2*-Wild.1d before introducing the mutation is basically the same protein as the variant which has the highest binding capability to IgG-Fab in Patent Document 2. The C-terminal and several residues at the N-terminal of the above-described GB2*-Wild.1d are different from the above-described variant due to the difference of the expression/preparation condition of a protein; however, the GB2*-Wild.1d has approximately similar binding activity to the variant, as the association constant of the variant described in Patent Document 2 is 1.6×10⁶ [1/M], which is an inverse number of 0.61×10⁻⁶ [M] described in Table 1 of Patent Document 2 as a dissociation constant, and the difference between the value and the association constant shown in Table 2 is about 1.4 times. It is a surprising result that the variant obtained by the present invention has higher binding capability to IgG-Fab than GB2*-Wild.1d of which binding capability is the highest in Patent Document 2 and the improvement degree of the binding force by introducing the mutation is superior to the contents of Patent Document 2.

In addition, in Patent Document 2, the mutation to substitute the 15^{th} position by Phe is introduced. However, it is a surprising unpredictable result that the substitution mutation by Phe does not produce an excellent effect to improve IgG-Fab binding force but the substitution mutation by Tyr or Trp as the same aromatic amino acid produces such an excellent effect. It is also a surprising result that the mutation at the 2^{nd} position has a profound effect on the binding to Fab although the a carbon in the main chain at the 2^{nd} position in the IgG-binding domain of SpG is located far from the interaction interface between the domain and a Fab region in the publically-known crystal structure.

(3) Analysis of Affinity of SpG-β2 Variant for IgG-Fab

An affinity of GB2-E15W.1d prepared by introducing the mutation into wild SpG-β2 domain, i.e. GB2-Wild.1d, for IgG-Fab was evaluated in a similar manner to the above-described (2). However, the concentration of a protein solution was adjusted to 0.8 μM, bonding response curves at the time of adding the solution (association phase, for 1 minute) and after the addition (dissociation phase, for 2 minutes) were obtained, and a fitting analysis was carried out using one concentration of the solution.

As a result, an association constant K_{A} was calculated as 1.6×10⁶ [1/M]. Since it is known that the association constant of a protein corresponding to GB2-Wild.1d before introducing the mutation is 0.4×10⁶ [1/M], which is an inverse number of 2.57×10⁻⁶ [M] described in Table 1 of Patent Document 2 as a dissociation constant, the improvement effect of a binding force to IgG-Fab by introducing the mutation of E15W is assumed to be comparable to those in comparison with GB2*-Wild.1d as described above-described (2). It is therefore assumed that the improvement effects of a binding force of similar various IgG-binding domains of Protein G for IgG-Fab by introducing the mutation according to the present invention may be comparable to each other.

(4) Confirmation of Affinity of SpG-β2* Two Residues Variant for IgG-Fab

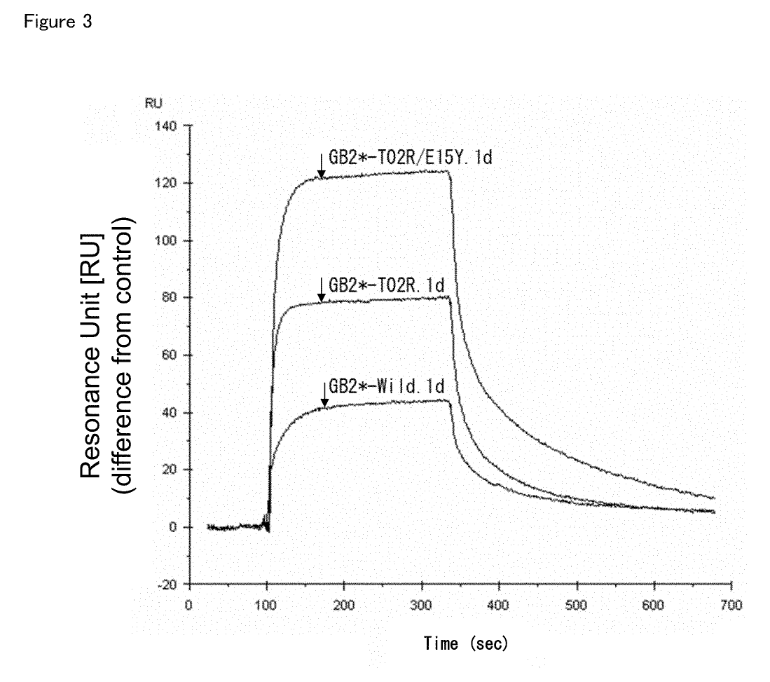

The affinity of GB2*-T02R/E15Y.1d (SEQ ID NO: 27), which is a two residues variant of SpG-β2*, for IgG-Fab was confirmed by using raw bonding response curve data in a similar manner of the above (2). In FIG. 3, the bonding response curve at the time of adding the protein solution having a concentration of 0.8 μM is demonstrated in addition to the bonding response curves of GB2*-Wild.1d and GB2*-T02R.1d having concentrations of 0.8 μM.

As shown in FIG. 3, it was confirmed that the binding response of GB2*-T02R/E15Y.1d is higher than those of GB2*-Wild.1d and GB2*-T02R.1d under the same concentration. It was found from the result that an improvement effect on a binding force to IgG-Fab can be produced even when two kinds of mutations according to the present invention are simultaneously introduced.

1. A Fab region-binding peptide, selected from the following (1) to (3):
(1) a Fab region-binding peptide comprising an amino acid sequence of SEQ ID NO: 1 derived from β1 domain of protein an amino acid sequence of SEQ ID NO: 2 derived from β2 domain of protein G or an amino acid sequence of SEQ ID NO: 3 derived from β2 domain variant of protein having one or more amino acid substitution mutations selected from a mutation of substituting the amino acid residue at the 2^{nd} position with Arg and a mutation of substituting the amino acid residue at the 15^{th} position with Tyr or Trp;
(2) a Fab region-binding peptide comprising the amino acid sequence specified in the (1) having deletion, substitution and/or addition of one or more amino acid residues in a region except for the 2^{nd} position and the 15^{th} position, and having higher binding force to a Fab region of an immunoglobulin G than a binding force before introducing the amino acid substitution mutation;
(3) a Fab region-binding peptide comprising an amino acid sequence having a sequence identity of 80% or more with the amino acid sequence specified in the (1), and having higher binding force to a Fab region of an immunoglobulin G than a binding force before introducing the amino acid substitution mutation, provided that the amino acid substitution mutation specified in the (1) at one or more positions selected from the 2^{nd} position and the 15^{th} position is not further mutated in (3).

2. The Fab region-binding peptide according to claim 1, wherein a position of the substitution is one or more positions selected from the 6^{th} position, the 7^{th} position, the 10^{th} position, the 13^{th} position, the 18^{th} position, the 19^{th} position, the 21^{st} position, the 24^{th} position, the 28^{th} position, the 29^{th} position, the 30^{th} position, the 31^{st} position, the 33^{rd} position, the 35^{th} position, the 39^{th} position, the 40^{th} position, the 42^{nd} position and the 47^{th} position in the amino acid sequence specified in the (2).

3. The Fab region-binding peptide according to claim 1, wherein a position of the deletion and/or addition is N-terminal and/or C-terminal in the amino acid sequence specified in the (2).

4. The Fab region-binding peptide according to claim 1, wherein the sequence identity is 90% or more in the amino acid sequence specified in the (3).

5. The Fab region-binding peptide according to claim 1, wherein the amino acid residue at the 15^{th} position is substituted with Tyr in the amino acid sequence specified in the (1).

6. A Fab region-binding peptide multimer, comprising two or more Fab region-binding peptides according to claim 1 as domains, wherein the Fab region-binding peptides are connected one another.

7. An affinity separation matrix, wherein the Fab region-binding peptide according to claim 1 or the Fab region-binding peptide multimer according to claim 6 is immobilized on a water-insoluble carrier.

8. A method for producing a protein comprising a Fab region, comprising the steps of:
contacting a liquid sample comprising the protein comprising the Fab region with the affinity separation matrix according to claim 7; and
separating the protein comprising the Fab region adsorbed on the affinity separation matrix from the affinity separation matrix.

9. A DNA, encoding the Fab region-binding peptide according to claim 1.

10. A vector, comprising the DNA according to claim 9.

11. A transformant, transformed by the vector according to claim 10.

12. The Fab region-binding peptide according to claim 1, wherein the amino acid residue at the 2^{nd} position is substituted with Arg in the amino acid sequence specified in the (1).

13. The Fab region-binding peptide according to claim 1, wherein the amino acid residue at the 15^{th} position is substituted with Trp in the amino acid sequence specified in the (1).
